(19) United States
(12) Patent Application Publication
(10) Pub. No.: US 2021/0137961 A1
(43) Pub. Date: May 13, 2021

(54) Compositions and Methods for Treating Dry Eye Diseases

(71) Applicant: CEMPRA PHARMACEUTICALS, INC., Chapel Hill, NC (US)

(72) Inventor: Prabhavathi Fernandes, Chapel Hill, NC (US)

(21) Appl. No.: 16/602,279

(22) PCT Filed: Mar. 7, 2018
(86) PCT No.: PCT/US2018/021304

§ 371 Date: Sep. 9, 2019

Related U.S. Application Data
(60) Provisional application No. 62/467,973, filed on Mar. 7, 2017.

(51) Int. Cl. A61K 31/7056; A61K 9/00; A61K 47/02; A61K 47/18; A61K 47/12; A61K 47/36; A61K 9/08; A61K 47/38; A61P 29/00; A61K 47/14; A61P 27/04

(52) U.S. Cl. CPC A61K 31/7056; A61K 9/0048; A61K 47/02; A61K 47/183; A61K 47/12; A61P 27/04; A61K 47/186; A61K 9/08; A61K 47/38; A61P 29/00; A61K 47/14; A61K 47/36

(57) ABSTRACT

Described herein are pharmaceutical compositions adapted for the topical administration of macrolide antibiotics, and uses thereof in the treatment of dry eye diseases.

### CROSS-REFERENCE TO RELATED APPLICATIONS

This application claims the benefit under 35 U.S.C. § 119(e) of U.S. Provisional Application Ser. No. 62/467,973 filed Mar. 7, 2017, the disclosure of which is incorporated herein by reference in its entirety.

### TECHNICAL FIELD

The invention described herein pertains to pharmaceutical compositions adapted for the topical administration of macrolide antibiotics, such as triazole-containing and fluoro ketolide antibiotics. The invention described herein also pertains to methods for their use in the treatment of dry eye diseases.

### BACKGROUND AND SUMMARY OF THE INVENTION

Macrolide antibiotics, characterized by a large lactone ring to which are attached one or more deoxy sugars, usually cladinose and desosamine, are antimicrobial drugs that are active against aerobic and anaerobic gram-positive cocci and are prescribed for the treatment of a number of infections, including respiratory tract and soft tissue infections. The macrolides, which belong to the polyketide class of natural products, function by reversibly binding to the 505 subunit of the bacterial ribosome, blocking protein synthesis and preventing bacterial growth and reproduction. Although this action is primarily bacteriostatic, certain fluoroketolides and triazole-containing macrolides are bactericidal.

Erythromycin (Ery) and the semi-synthetic derivatives azithromycin (Azi) and clarithromycin (Clari) are among the marketed macrolide antibiotics. Telithromycin (Teli) and cethromycin belong to the ketolide group of antibiotics. Oral administration has been accomplished for many macrolides and ketolides; however, the corresponding topical administration of known macrolides and ketolides, especially approved macrolides such as erythromycin, clarithromycin, telithromycin, and azithromycin, has been hampered by the requirement for repeat dosing because of the low bioavailability and/or short half-life of the macrolide or ketolide in the target tissue (see, for example, Bowman et al., J Ocular Pharm Therap 25(2):133-139 (2009)). In particular, tearing has been reported to have the effect of washing away the administered drug from the pre-corneal area, and decreasing the bioavailability. For example, commonly most topical regimens using known antibiotics such as gentamycin and erythromycin must be administered frequently with application rates of 4-6 times daily sometimes being required to produce effective drug levels in target ocular tissues.

Without being bound by theory, it is believed herein that the effective treatment of eye diseases depends on sufficient drug being absorbed into the diseases eye tissue, such as the cornea, conjunctivae, and eyelid tissue. The need for repeated dosing has resulted in limitations on use, and issues with patient compliance.

Accordingly, a need exists for alternative ophthalmic formulations of macrolides, especially ketolides, that may be administered directly to the eye. Described herein are pharmaceutical compositions adapted for the topical administration of the triazole-containing ketolide antibiotics and fluoro derivatives thereof, such as solithromycin and related compounds, as well as methods for their use in the treatment of dry eye diseases.

It has been unexpectedly discovered that macrolide and ketolide compounds described herein are useful for treating dry eye diseases. It has been discovered that compounds described herein positively affect lysosome and lipid accumulation and differentiation of meibomian glands. Compounds described herein act directly on meibomian glands to stimulate lysosome generation and lipid accumulation, the formation of lamellar bodies, and overall maturation and differentiation of meibomian glands. Without being bound by theory it is believed that this stimulation arises from directly induced phospholipidosis in meibomian gland epithelial cells. It has also been unexpectedly discovered that compounds described herein induce phospholipodosis without causing apoptosis. Other compounds, such as gentamicin and azithromycin induce phospholipodosis with concomitant apoptosis.

It has also been discovered that compounds described herein are efficacious in causing the differentiation of meibomian gland epithelial cells. Compounds described herein are useful in treating meibomian gland dysfunction, and various dry eye diseases. In contrast, most other macrolides and ketolides, such as erythromycin, clarithromycin, cethromycin, and telithromycin, do not cause clinically significant meibomian gland differentiation.

Meibomian glands, also known as tarsal glands, are a special kind of sebaceous gland at the rim of the eyelids inside the tarsal plate. Meibomian glands are responsible for the supply of meibum, an oily substance that prevents evaporation of the eye's tear film. Meibum also prevents tear spillage onto the cheek, trapping tears between the oiled edge and the eyeball, also allowing the lids to make an airtight seal when closed. There are approximately 50 glands on the upper eyelids and 25 glands on the lower eyelids. Normally, meibomian glands generate abundant lipids that accumulate in lysosomes, which are secreted, termed meibum, in a holocrine manner into lateral ducts, and are ultimately released onto the ocular surface. Meibum provides a clear optical surface for the cornea, interferes with bacterial colonization, and retards tear overflow. Meibum also promotes the stability and prevents the evaporation of the tear film, thereby playing a critical role in the health of the ocular surface.

Dysfunctional meibomian glands often cause dry eyes, one of the more common eye conditions. They may also contribute to blepharitis. Inflammation of the meibomian glands, also known as meibomitis, meibomian gland dysfunction, or posterior blepharitis, causes the glands to be obstructed by thick waxy secretions. Besides leading to dry eyes, such waxy obstructions can be degraded by bacterial lipases, resulting in the formation of free fatty acids, which further irritate the eyes and exacerbate the dry eye condition. Meibomian gland dysfunction may lead to punctate keratopathy. Meibomian gland dysfunction is more often seen in women and is regarded as the main cause of dry eye disease in women.

Meibomian gland dysfunction is caused primarily by hyperkeratinization of the terminal duct epithelium and reduced secretion quality, and leads to cystic dilatation of glandular ducts, acinar cell death, and lipid deficiency. The end result is a dry eye disease, characterized by a vicious cycle of tear film hyperosmolarity and ocular surface stress, and leading to increased friction, inflammation and damage to the eye.

Keratoconjunctivitis sicca (KCS), also called dry eye syndrome (DES) or keratitis sicca, is an eye disease caused by eye dryness, which, in turn, is caused by either decreased tear production or increased tear film evaporation. KCS and DES occur in humans and some other animals. KCS is the more common eye disease, affecting 5-6% of the population. Prevalence rises to 6-10% in postmenopausal women, and can be as high as 34% in the elderly. Most persons affected by dry eyes experience mild irritation with no long-term effects. However, if the condition is left untreated or becomes severe, it can produce complications that can cause eye damage, resulting in impaired vision, and in some case, loss of vision.

In addition, prolonged dry eyes can lead to the formation of tiny abrasions on the surface of the eyes. In advanced cases, the epithelium undergoes pathologic changes, including squamous metaplasia and loss of goblet cells. In even more severe cases, thickening of the corneal surface, corneal erosion, punctate keratopathy, epithelial defects, corneal ulceration (sterile and infected), corneal neovascularization, corneal scarring, corneal thinning, and even corneal perforation can occur.

It has been unexpectedly discovered herein that triazole-containing macrolide and ketolide antibiotics and fluoro derivatives thereof, such as solithromycin and related compounds, may be administered topically to the eye. It is also discovered herein that triazole-containing macrolide and ketolide antibiotics and fluoro derivatives thereof, such as solithromycin and related compounds, may be administered once daily (q.d.), and such dosing is effective in treating dry eye diseases. It has also been discovered that triazole-containing macrolide and ketolide antibiotics and fluoro derivatives thereof, such as solithromycin and related compounds, are effective anti-inflammatory agents and as such effective in treating the inflammatory aspects of dry eye diseases. It has also been discovered herein that triazole-containing macrolide and ketolide antibiotics and fluoro derivatives thereof, such as solithromycin and related compounds, undergo rapid corneal penetration leading to unexpectedly improved bioavailability. It has been reported that most eye-drops experience rapid clearance from ocular surface via naso-lacrimal drainage, where ˜95% of each eye-drop is lost within 1 hour. Rapid corneal penetration provides for less frequent dosing regimens because of the lower clearance mechanisms such as tearing that affect dosing. It is also discovered herein that compounds described herein exhibit long half-lives in target tissues. It is also discovered herein that triazole-containing macrolide and ketolide antibiotics and fluoro derivatives thereof, such as solithromycin and related compounds, do not cause clinically significant irritation of ocular tissues when administered topically. It is also discovered herein that triazole-containing macrolide and ketolide antibiotics and fluoro derivatives thereof, such as solithromycin and related compounds, exhibit high solution stability even during long term storage. It is also discovered herein that triazole-containing macrolide and ketolide antibiotics and fluoro derivatives thereof, such as solithromycin and related compounds, exhibit ultimately higher comparative concentrations in diseased tissues. It is also discovered herein that triazole-containing macrolide and ketolide antibiotics and fluoro derivatives thereof, such as solithromycin and related compounds, exhibit substantially higher antibacterial efficacy at lower pH than conventional antibiotics. It is appreciated herein that lacrimal conditions may be more acidic than other tissues, and therefore, effective treatment will be compromised by compounds that lose potency at lower pH. It is also appreciated herein that inflammation may cause the affected tissues to be more acidic than other tissues, and therefore, effective treatment will be compromised by compounds that lose potency at lower pH.

International patent application, publication number WO 2004/080391, incorporated herein by reference, describes a family of triazole-containing and macrolide and ketolide antibiotics. Illustrative of those antibiotics are compounds of the formula:

[chemical structure]

and pharmaceutically acceptable salts, hydrates, solvates, esters, and prodrugs thereof, wherein:

R¹⁰ is hydrogen or acyl;

W is H, F, Cl, Br, I, or OH;

A is CH₂, C(O), C(O)O, C(O)NH, S(O)₂, S(O)₂NH, C(O)NHS(O)₂;

B is C₀ to C₁₀ alkylene; or B is C₂ to C₁₀ alkenylene; or B is C₂ to C₁₀ alkynylene; or B is or C₄ to C₁₀ alkenylalkynylene; and

C is aryl, heteroaryl, arylalkyl, or heteroarylalkyl, each of which is optionally substituted.

Further illustrative of those compounds is CEM-101, Chemical Abstracts Registry Number 760981-83-7 and also known as solithromycin or Soli, and having the following structure:

[chemical structure]

and pharmaceutically acceptable salts, hydrates, solvates, esters, and prodrugs thereof.

### DETAILED DESCRIPTION

In one illustrative embodiment of the invention described herein, compounds, compositions, formulations, kits, and methods and uses thereof are described herein for treating dry eye diseases.

In another embodiment, compounds, compositions, and methods are described herein for treating eye diseases, including eye diseases that have both a bacterial and inflammatory component.

Illustrative eye diseases treatable by the compounds, compositions, formulations and methods described herein, include inflammatory conditions, inflammatory conjunctivitis, blepharitis, including inflammatory blepharitis, posterior blepharitis, and anterior blepharitis, such as of the eye lid, under the lid, of the conjunctivae, and the like, dry eye syndrome, keratoconjunctivitis sicca (KCS), dysfunctional tear syndrome, lacrimal keratoconjunctivitis, evaporative tear deficiency, aqueous tear deficiency, and LASIK-induced neurotrophic epitheliopathy (LNE) and the like.

Soli and related triazole-containing macrolides and ketolides are highly potent compounds that retain activity against drug-resistant strains, including showing potent activity against S. pneumoniae, as well as having an extended spectrum of activity against community acquired-methicillin resistant Staphylococcus aureus (CA-MRSA), enterococci, M. avium, and showing efficacy in animal models of malaria. They are also active against atypical bacteria, such as Leginella, Mycoplasma and Ureaplasma, and against gonococci and other organisms that cause genitourinary tract infections. Soli has been observed to often be 8-16 times more potent than azithromycin and is active against azithromycin-resistant strains. Without being bound by theory, it is believed herein that the activity of Soli and related compounds against resistant strains may be driven by their ability to bind to three sites on the bacterial ribosome, compared to one or two sites for currently available macrolides.

In another embodiment, the compositions described herein are efficacious against one or more of the following pathogens Corynebacterium spp., including Corynebacterium diphtheriae, Haemophilus influenzae, Streptococcus pneumoniae, Staphylococcus aureus, CA-MRSA, Chlamydophila pneumoniae Chlamydia trachomatis, Haemophilus parainfluenzae, Legionella pneumophila, Listeria monocytogenes, Moraxella catarrhalis, Mycobacterium avium, Mycoplasma hominis, Mycoplasma pneumoniae, Neisseria gonorrhoeae, Peptostreptococcus spp., Ureaplasma urealyticum, Viridans group streptococci, Streptococcus mitis, Streptococcus pyogenes, Streptococcus agalactiae, Streptococci (Groups C, F, G), and the like.

In another embodiment, pharmaceutical compositions are described adapted for topical administration directly to the surface of the eye, comprising one or more compounds selected from the group consisting of triazole-containing macrolides and ketolides, and fluoroketolides, such as Soli and related compounds, and combinations thereof. In another embodiment, the compound is a triazole-containing fluoroketolide. In another embodiment, the composition is a concentrate. In another embodiment, the composition further comprises one or more acidifying agents. In another embodiment, the composition further comprises one or more alkalizing agents. In another embodiment, the composition further comprises one or more aqueous diluents. In another embodiment, the composition further comprises one or more stabilizers. In another embodiment, the composition further comprises one or more anti-oxidants. In another embodiment, the composition further comprises one or more excipients. In another embodiment, the composition further comprises one or more buffering agents.

Several illustrative embodiments of the invention are described by the following clauses:

1. A method for treating one or more dry eye diseases in a host animal, the method comprising the step of topically administering to an eye of the host animal an effective amount of a composition comprising one or more compounds of the formula

[chemical structure]

or a pharmaceutically acceptable salt thereof, wherein:

R¹⁰ is hydrogen or acyl;

W is H, F, Cl, Br, I, or OH;

A is CH₂, C(O), C(O)O, C(O)NH, S(O)₂, S(O)₂NH, or C(O)NHS(O)₂;

B is C₀ to C₁₀ alkylene, C₂ to C₁₀ alkenylene, C₂ to C₁₀ alkynylene, or C₄ to C₁₀ alkenylalkynylene; and

C is aryl, heteroaryl, arylalkyl, or heteroarylalkyl, each of which is optionally substituted.

2. Use of one or more compounds of the formula

[chemical structure]

or a pharmaceutically acceptable salt thereof, in the manufacture of a medicament for treating one or more dry eye diseases in a host animal, wherein:

R¹⁰ is hydrogen or acyl;

W is H, F, Cl, Br, I, or OH;

A is CH₂, C(O), C(O)O, C(O)NH, S(O)₂, S(O)₂NH, or C(O)NHS(O)₂;

B is C₀ to C₁₀ alkylene, C₂ to C₁₀ alkenylene, C₂ to C₁₀ alkynylene, or C₄ to C₁₀ alkenylalkynylene; and

C is aryl, heteroaryl, arylalkyl, or heteroarylalkyl, each of which is optionally substituted.

3. A pharmaceutical composition for treating one or more dry eye diseases in a host animal, the composition comprising an effective amount of one or more compounds of the formula

[chemical structure]

or a pharmaceutically acceptable salt thereof, wherein:

R¹⁰ is hydrogen or acyl;

W is H, F, Cl, Br, I, or OH;

A is CH₂, C(O), C(O)O, C(O)NH, S(O)₂, S(O)₂NH, or C(O)NHS(O)₂;

B is C₀ to C₁₀ alkylene, C₂ to C₁₀ alkenylene, C₂ to C₁₀ alkynylene or C₄ to C₁₀ alkenylalkynylene; and

C is aryl, heteroaryl, arylalkyl, or heteroarylalkyl, each of which is optionally substituted.

4. The method, use, or composition of any one of the preceding clauses further comprising one or more carriers, diluents, or excipients, or a combination thereof.

5. The method, use, or composition of any one of the preceding clauses wherein the disease is keratoconjunctivitis sicca.

6. The method, use, or composition of any one of the preceding clauses wherein the disease is dry eye syndrome.

7. The method, use, or composition of any one of the preceding clauses wherein the disease is keratitis sicca.

8. The method, use, or composition of any one of the preceding clauses wherein the disease is keratoconjunctivitis.

9. The method, use, or composition of any one of the preceding clauses wherein the disease is blepharitis.

10. The method, use, or composition of any one of the preceding clauses wherein the disease is blepharoconjunctivitis.

11. The method, use, or composition of any one of the preceding clauses wherein A is CH₂.

12. The method, use, or composition of any one of the preceding clauses wherein B is C₀ to C₁₀ alkylene, or C₂ to C₁₀ alkylene, or C₂ to C₆ alkylene, or C₃ to C₆ alkylene, or C₃ to C₅ alkylene, C₃ to C₄ alkylene, or C₃ alkylene.

13. The method, use, or composition of any one of the preceding clauses wherein B is of the formula (CH₂)ₙ, where n is an integer selected from 2-10, 2-6, 3-6, 3-5, 3-4, 4, or 3.

14. The method, use, or composition of any one of the preceding clauses wherein C is optionally substituted aryl.

15. The method, use, or composition of any one of the preceding clauses wherein C is substituted aryl.

16. The method, use, or composition of any one of the preceding clauses wherein C is optionally substituted heteroaryl.

17. The method, use, or composition of any one of the preceding clauses wherein C is substituted heteroaryl.

18. The method, use, or composition of any one of the preceding clauses wherein C is unsubstituted heteroaryl.

19. The method, use, or composition of any one of the preceding clauses wherein C is aminoaryl, or an amino prodrug thereof.

20. The method, use, or composition of any one of the preceding clauses wherein C is aminoaryl.

21. The method, use, or composition of any one of the preceding clauses wherein C is amidoaryl.

22. The method, use, or composition of any one of the preceding clauses wherein C is optionally substituted phenyl.

23. The method, use, or composition of any one of the preceding clauses wherein C is substituted phenyl.

24. The method, use, or composition of any one of the preceding clauses wherein C is aminophenyl, or an amino prodrug thereof.

25. The method, use, or composition of any one of the preceding clauses wherein C is aminophenyl.

26. The method, use, or composition of any one of the preceding clauses wherein C is amidophenyl.

27. The method, use, or composition of any one of the preceding clauses wherein C is 3-aminophenyl, or an amino prodrug thereof.

28. The method, use, or composition of any one of the preceding clauses wherein C is 3-aminophenyl.

29. The method, use, or composition of any one of the preceding clauses wherein C is 3-amidophenyl.

30. The method, use, or composition of any one of the preceding clauses wherein W is hydrogen.

31. The method, use, or composition of any one of the preceding clauses wherein W is F.

32. The method, use, or composition of any one of the preceding clauses wherein R¹⁰ is hydrogen.

33. The method, use, or composition of any one of the preceding clauses wherein R¹⁰ is acyl, such as optionally substituted alkylacyl or arylacyl, such as optionally substituted benzoyl or benzoyl.

34. The method, use, or composition of any one of the preceding clauses wherein at least one compound is solithromycin, or a salt thereof, or an amino prodrug of any of the foregoing. It is to be understood that the source of solithromycin may be of any form or mixture thereof, including a solution, suspension, or solid. Solid forms may be an amorphous form or one or more crystalline forms, or mixtures thereof. Illustrative crystal forms of solithromycin are described in PCT international publication No. 2011/119604, the disclosure of which is incorporated herein by reference.

35. The method, use, or composition of any one of the preceding clauses wherein at least one compound is solithromycin, or a salt thereof, such as the hydrochloric acid or tartaric acid salt thereof.

36. The method, use, or composition of any one of the preceding clauses wherein at least one compound is solithromycin, or an amino acid salt thereof, such as the aspartic acid, glutamic acid, or histidine salt thereof.

37. The method, use, or composition of any one of the preceding clauses wherein at least one compound is solithromycin.

38. The method, use, or composition of any one of the preceding clauses wherein at least one carrier is water.

39. The method, use, or composition of any one of the preceding clauses wherein at least one carrier is ultrapure water.

40. The method, use, or composition of any one of the preceding clauses wherein the composition includes boric acid or a salt thereof. Illustratively, the boric acid or salts thereof are in the range from about 0.02% to about 2.0%, from about 0.05% to about 1.0%, from about 0.05% to about 0.25%, from about 0.1% to about 0.2%, from about 0.1% to about 0.15%, or about 0.15% by weight. It has been discovered herein that boric acid, and salts thereof, stabilize formulations containing the compounds of formula (I). Without being bound by theory, it is believed herein that boric acid, and salts thereof, stabilize formulations containing the compounds of formula (I) via complexation, which may decrease the oxidation potential of the compounds. It has been observed that other formulations of the compounds of formula (I) degrade by oxidation. Without being bound by theory, it is believed herein that the oxidation degradation products are N-oxides.

41. The method, use, or composition of any one of the preceding clauses wherein the composition includes a metal chelating agent, such as EDTA or a salt thereof. Illustratively, the metal chelating agent, such as EDTA or a salt thereof, is in the range from about 0.01% to about 0.1%, such as about 0.05%, by weight. It has been unexpectedly discovered that mixtures of boric acid and salts thereof and chelating agents, such as EDTA, stabilize formulations described herein better than when boric acid and salts thereof are used alone. The observed stabilization improvement is unexpected because the use of metal chelating agents, such as EDTA or a salt thereof, alone does not appear to affect stability either positively or negatively.

42. The method, use, or composition of any one of the preceding clauses wherein the composition includes one or more polyethylene glycols (PEGs) esters. Illustratively, the PEG esters are selected from PEG castor oil, such as PEG35 castor oil, or PEG stearate, such as PEG40 stearate, or any combination of the foregoing. Alternatively, the PEGs consist of or consist essentially of PEG400, PEG35 castor oil, or PEG40 stearate, or a combination thereof. It has been observed that compounds of formula (I) have limited solubility in aqueous systems at pH levels greater than about 4, and that limited solubility decreases as the pH approaches neutrality. It has been unexpectedly discovered that PEG esters solubilize the compounds of formula (I) and provide concentrated solutions of at least about 1% by weight, which is approximately 10 mg/mL.

43. The method, use, or composition of any one of the preceding clauses wherein the composition includes one or more PEG esters totaling at about 20%, about 18%, about 16%, about 15%, about 14%, about 13%, about 12%, about 11%, about 10%, about 9%, about 8%, or about 7%, or in the range from about 5% to about 15%, from about 5% to about 14%, from about 5% to about 13%, from about 5% to about 12%, from about 5% to about 11%, or from about 5% to about 10% by weight. Alternatively, the composition includes one or more PEG esters in the range from about 8% to about 15%, from about 8% to about 14%, from about 9% to about 14%, from about 9% to about 13%, from about 10% to about 13%, from about 11% to about 13%, from about 9% to about 12%, from about 10% to about 12%, from about 11% to about 12%, or at about 12% by weight.

44. The method, use, or composition of any one of the preceding clauses wherein the composition includes two or more PEG esters totaling an amount selected from the preceding clause. Illustratively, one PEG ester is saturated, and the one PEG ester is unsaturated, hydroxylated, or unsaturated and hydroxylated. Illustratively, the ratio of saturated PEG ester to other PEG ester is in the range from about 5:1 to about 1:5, in the range from about 4:1 to about 1:4, in the range from about 3:1 to about 1:3, in the range from about 2:1 to about 1:2, or in the range from about 3:2 to about 2:3. Alternatively, the ratio of saturated PEG ester to other PEG ester is in the range from about 5:1 to about 1:1, in the range from about 4:1 to about 1:1, in the range from about 3:1 to about 1:1, in the range from about 2:1 to about 1:1, or in the range from about 3:2 to about 1:1. Illustratively, the amount of saturated PEG ester, such as PEG stearate or PEG40 stearate, is greater than the amount of the other PEG ester that is unsaturated, hydroxylated, or unsaturated and hydroxylated, such as PEG castor oil or PEG35 castor oil.

45. The method, use, or composition of any one of the preceding clauses wherein the composition includes an osmolality modifying agent, also known as a tonicity agent, such as glycerin, polyethylene glycol, propylene glycol, trehalose, mannitol, sucrose, and the like. Illustratively, the osmolality modifying agent is a PEG, and the like. Illustratively, the PEG is included at about 5% or less, about 4% or less, about 3% or less, about 2% or less, in the range from about 0.1 to about 1.9%, about 0.1 to about 1.5%, about 0.5 to about 1.5%, about 0.8 to about 1.2%, about 0.9 to about 1.1%, or about 1% PEG by weight. Illustratively, the PEG is PEG400. It has been unexpectedly discovered that certain osmolality modifying agents negatively affect the stability of formulations described herein, or cause precipitation of the compounds of formula (I). Illustratively, the osmolality modifying agent is not a poloxamer or a cyclodextrin. Illustratively, the formulation has a physiologically acceptable osmolality, such as an osmolality of about 250-350 mOsm/kg, or about 280-300 mOsm/kg, about 280-320 mOsm/kg, about 285-320 mOsm/kg, about 290-320 mOsm/kg, or about 290-300 mOsm/kg.

It has been unexpectedly discovered that xanthan gum and related compounds are compatible with solithromycin, whereas other viscosity modifying agents are less so. It has also been unexpectedly discovered that formulations described herein that include a viscosifying agent, such as xanthan gum, exhibit greater efficacy against commensal bacterial overgrowth in the eye, such as bacterial overgrowth that accompanies blepharitis. Such formulations show efficacious exposure in the relevant tissues, including surface tissues such as the eyelids.

46. The method, use, or composition of any one of the preceding clauses wherein the composition includes a viscosifying agent, such as xanthan gum or an analog thereof, such as but not limited to locust bean gum, anionic polysaccharides, guar gum, and the like. Illustratively, the xanthan gum is in the range from about 0.01% to about 2%, from about 0.1% to about 1%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or about 0.3%, or about 0.2% by weight. It has been discovered herein that xanthan gum and chemically related compounds stabilize formulations containing the compounds of formula (I), and also provides increased viscosity. It has been observed that certain viscosifying agents are less compatible with the methods, uses, compositions, and/or formulations described herein and lead to instability and precipitation of the compounds of formula (I). For example, polycarbophil and Pluronics negatively affect the stability of the formulation, or facilitate precipitation of the compounds of formula (I). Illustratively, the viscosifying agent is substantially free of, or free of polycarbophil, polyacrylates, Carbopol, carboxymethyl cellulose, polyvinyl alcohol (PVA), polyvinyl pyrrolidone (PVP), poloxamers, or Pluronics, or a combination of the foregoing.

47. The method, use, or composition of any one of the preceding clauses wherein the composition includes a viscosifying agent, such as hyaluronic acid or a salt thereof, or an analog thereof. Illustratively, the hyaluronic acid or a salt thereof is in the range from about 0.01% to about 2%, from about 0.1% to about 1%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or about 0.2% by weight. It has been discovered herein that hyaluronic acid and salts thereof stabilize formulations containing the compounds of formula (I), and also provides increased viscosity. It has been observed that certain viscosifying agents are less compatible with the methods, uses, compositions, and/or formulations described herein and lead to instability and precipitation of the compounds of formula (I).

It has been unexpectedly discovered that hyaluronic acid and salts thereof are compatible with solithromycin, whereas other viscosity modifying agents from different chemical are less so.

48. The method, use, or composition of any one of the preceding clauses wherein the composition is substantially free of excipients selected from the group consisting of poloxamers, polyvinyl alcohols (PVAs), polyvinyl pyrrolidones (PVPs), polyacrylates, and combinations thereof.

It has been discovered herein that hyaluronic acid stabilizes formulations containing the compounds of formula (I), and also provide increased viscosity. Other conventional excipients are less compatible because their inclusion in the methods, uses, compositions, and/or formulations described herein accelerate the chemical degradation of the compounds of formula (I), and/or decrease the solubility of the compounds of formula (I).

49. The method, use, or composition of any one of the preceding clauses wherein the composition includes a buffer and/or acidifying agent. Illustratively, the buffer includes citric acid, or a salt or hydrate thereof, or a combination of any of the foregoing. Illustratively, the acidifying agents include, but are not limited to, ascorbic acid, and/or a tartaric acid, or a combination thereof. In another embodiment, the acidifying agent is a tartaric acid, such as L-tartaric acid. It has been observed that certain buffer agents are less compatible with the methods, uses, compositions, and/or formulations described herein and lead to instability and/or precipitation of the compounds of formula (I). For example, phosphates negatively affect the stability of the formulation, or facilitate precipitation of the compounds of formula (I). Illustratively, the buffer is substantially free of, or free of phosphate.

50. The method, use, or composition of any one of the preceding clauses wherein the composition includes a preservative. Illustrative preservatives include, but are not limited to, one or more benzalkonium chlorides, and the like. It is understood herein that the amount of preservative should be as low as possible. Illustrative amounts of preservatives are less than about 0.1%, 0.02%, 0.015%, 0.01%, 0.009%, 0.008%, 0.007%, 0.006%, 0.005%, 0.004%, 0.003%, 0.002%, 0.001%, and the like. Alternatively, formulations are described herein that are substantially free of, or free of preservatives. It has been unexpectedly discovered that the formulations described herein do not require any preservative.

51. The method, use, or composition of any one of the preceding clauses wherein the composition is substantially free of, or free of chloride.

52. The method, use, or composition of any one of the preceding clauses wherein the composition is substantially free of propylene glycol and/or polypropylene glycol.

53. The method, use, or composition of any one of the preceding clauses wherein the composition is substantially free of ethanol.

54. The method, use, or composition of any one of the preceding clauses wherein the composition is substantially free of PVP, poloxamer, such as Poloxamer 188, and/or alkyl aryl polyether alcohols, such as tyloxapol.

55. The method, use, or composition of any one of the preceding clauses wherein the composition includes excipients that are substantially free of peroxides and/or formaldehyde, such as excipients selected from the group consisting of PEG400, PEG35 castor oil, PEG40 Stearate, ultrapure water, and the like, where the excipient is substantially free of peroxides or formaldehyde.

56 The method, use, or composition of any one of the preceding clauses wherein the compound of formula (I) is present in the composition at a concentration in the range from about 0.1 to about 2.5 weight percent, from about 0.2 to about 2.0 weight percent, from about 0.5 to about 1.0 or from about 0.5 to about 1.1 weight percent, from about 0.6 to about 1.0, or from about 0.6 to about 1.1 weight percent, from about 0.7 to about 1.0 or from about 0.7 to about 1.1 weight percent, from about 0.8 to about 1.0 or from about 0.8 to about 1.1 weight percent, from about 0.9 to about 1.0, from about 0.9 to about 1.1 weight percent, or at about 1% by weight.

5572. The method, use, or composition of any one of the preceding clauses that is substantially oxygen free or oxygen free, such as including a substantially oxygen free or oxygen free enveloping gas, such as nitrogen or argon gas. Illustratively, the composition or formulation of any one of the preceding clauses include less than about 10 ppm, about 6 ppm, about 5 ppm, or 4 ppm dissolved oxygen.

58. A package or a kit containing the formulation or composition of any one of the preceding clauses for use in the method of any one of the preceding clauses, and including instructions.

59. The package or kit of the preceding clause also including an applicator, such as a dropper. Illustratively, the dropper provides a drop between about 20 and about 40 mg, or between about 25 and about 35 mg.

60. The method, use, composition, or kit of any one of the preceding clauses adapted for once a day administration.

61. The method, use, or composition of any one of the preceding clauses wherein the host animal is a human.

In reciting the foregoing collection of clauses, it is to be understood that all possible combinations of features, and all possible subgenera and subcombination are described. In addition, it is to be understood that each of the foregoing clauses and embodiments may be combined with any other embodiment described herein in all possible combinations.

It is to be understood that the source of the compound described herein is from a variety of sources. With specific reference to solithromycin, the compositions described herein may be prepared from amorphous or crystalline material, which may be in any case a salt form, hydrate, solvate, and the like. It is to be understood that the purity of the source of the compound, such as solithromycin, is to be considered. Illustrative salt forms of the compounds described herein, such as solithromycin, include but are not limited to HCl, tartrate, lactate, lactobionate, oxalate, acetate, trifluoroacetate, fumarate, and the like.

The compounds described herein, including solithromycin, also known as CEM-101 and OP-1068, may be prepared as described in WO 2004/080391, WO 2009/055557, US 20130066056, WO 2011/146829, or by other conventional procedures, or by a procedure analogous to one of the described or known procedures.

In another embodiment, the composition includes one or more components selected from, but not limited to, one or more amino acids, such as histidine or a salt thereof, glutamic acid or a salt thereof, or aspartic acid or a salt thereof, and any combination thereof.

In another embodiment, the composition includes or also includes one or more components selected from, but not limited to, glycine, or a salt thereof, one or more carboxylic acids, such as tartaric acid or a salt thereof, acetic acid or a salt thereof, citric acid or a salt or hydrate thereof, or lactic acid or a salt thereof, one or more sugars, carbohydrates, or polyhydroxy compounds, such as mannitol, and combinations thereof.

It has been unexpectedly discovered that sterile filtration of xanthan gum solutions in the range of 0.6, 0.3, and 0.15% (w/w) xanthan gum in water with a 0.2 μm PES filter membrane was difficult. It was unexpectedly discovered that in xanthan gum solutions that included salts, e.g. citrate salts could be filtered through a 0.2 μm PES filter membrane.

In another embodiment, the composition is one wherein the alkalizing agents include, but are not limited to sodium hydroxide, and the like.

In another embodiment, the pH of the composition is about 4 or greater. In another embodiment, the pH of the composition is about 4.5 or greater. In another embodiment, the pH of the composition is about 8 or less, about 7 or less, about 6.5 or less, or about 6 or less. In another embodiment, the pH of the composition is between about 4 and about 6.5. In another embodiment, the pH of the composition is between about 4.5 and about 6.5, between about 5 and about 6.5, between about 5.5 and about 6.5, or between about 5.7 and about 6.3. It is to be understood that the relative amount of alkalizing agent may dependent upon the amount of acidifying agent, or ratio of the compound of formula (I) to the acidifying agent. It is to be understood herein that minimal buffering may be used in formulations that include low pH.

As used herein, the term “about” illustratively refers to a range of ±0.3, ±0.2, or ±0.1 with reference to a parameter or value described herein.

As used herein, the term “about” illustratively refers to a range of ±15%, ±10%, ±7.5%, ±5%, ±2.5%, or ±1% with reference to a parameter or value described herein. In another embodiment, the compositions described herein exhibit low viscosity.

It has been reported that high viscosity is necessary to ensure that the antibacterial agent will have a sufficiently long residence time on the eye tissue for efficacy, or to allow tissue absorption, or to decrease loss due to tearing. However, such viscous solutions have reportedly negatively affected patient compliance. The compositions described herein have been unexpectedly found to exhibit rapid tissue penetration such that highly viscous solutions are not necessary.

Illustratively, the viscosity is in the high range from about 200 cP to about 2200 cP, or about 400 cP to about 2200 cP, or about 600 cP to about 2200 cP, or about 600 cP to about 2000 cP, or about 800 cP to about 2000 cP, or about 1000 cP to about 2000 cP, or about 1100 cP to about 2000 cP, or about 1200 cP to about 2000 cP, or about 1200 cP to about 2000 cP, or about 1300 cP to about 2000 cP, or about 1400 cP to about 2000 cP, or about 1500 cP to about 2000 cP. Alternatively, the viscosity is in the midrange from about 10 cP to about 1000 cP, or about 10 cP to about 800 cP, or about 10 cP to about 600 cP, or about 10 cP to about 400 cP, or about 100 cP to about 400 cP, or about 200 cP to about 400 cP, or about 300 cP to about 400 cP. Alternatively, the viscosity is in the low range from about 1 cP to about 100 cP, or about 1 cP to about 80 cP, or about 1 cP to about 60 cP, or about 1 cP to about 40 cP, or about 1 cP to about 20 cP, or about 1 cP to about 10 cP, or about 1 cP to about 5 cP.

It has been discovered herein that conventional macrolides at low pH exhibit shorter storage stability life than the compounds described herein. Without being bound by theory, it is believed herein that the shorter storage life may be due to the loss of the cladinose sugar.

In another embodiment of the composition herein is one further comprising an anti-oxidant. In one embodiment, the anti-oxidant is 1-thioglycerol (also referred to as monothioglycerol or MTG). In one embodiment, the concentration of the anti-oxidant is about 5 mg/mL.

In another embodiment, the compositions include a stabilizing agent. Illustrative stabilizing agents include antioxidants, chelating agents, and the like, such as but not limited to ascorbic acid, cysteine, glutathione, sodium bisulphite, sodium metabisulphite, and the like. Illustrative concentrations of stabilizers, including anti-oxidants include, but are not limited to, 0.05%, 0.15%, 0.25%, 0.5% and 1.0%, and the like. Illustrative levels of additional anti-oxidants excluding EDTA include, but are not limited to, 0.25%, 0.5% and 1.0%, and the like.

In another embodiment, the compositions include a photostabilizing agent, such as a photo-oxidation stabilizing agent.

In another embodiment of the composition herein is one further comprising a surfactant. Illustrative surfactants include, but are not limited to, Tween 80, and the like, Polysorbate 80, polyoxyethylene hydrogenated castor oil 60, polyoxyl 35 castor oil, macrogol 4000, lecithin, sucrose ester, polyoxyethylene alkyl ether, polyoxyl stearate, polyoxyethylene polyoxypropylene glycol, vitamin E and/or one or more vitamin E derivatives, such as d-alpha tocopheryl polyethylene glycol 1000 succinate (TPGS). and the like. The concentration of the surfactant is illustratively 0.001% to about 0.5%.

In one embodiment, the pharmaceutical composition described herein is administered directly. In another embodiment, the pharmaceutical composition described herein is administered after further dilution.

A further embodiment is a single dose or multiple dose pharmaceutical dosage unit comprising a therapeutically effective amount of a pharmaceutical composition adapted for topical ophthalmic administration as described herein.

In another embodiment, the processes described herein include the step of sterilizing the formulation. Sterilization may be accomplished by any conventional process step, including but not limited to, by radiation treatment, such as gamma radiation, autoclaving (terminal sterilization), such as at a temperature of about 100° C. to about 125° C., or at about 121° C., by filtration, such as filtration using SUPOR membrane filter (0.2 μm)—Hydrophilic Polyethersulfone, DURAPORE membrane filter (0.22 μm)—Polyvinylidene Fluoride (Hydrophilic), NYLON membrane filter (0.2 μm)—Nylon Hydrophilic, and the like.

Without being bound by theory, it is understood herein that the excipients may function as bulking agents, osmolality adjusting agents, tonicity adjusting agents, stabilizing agents, buffers, antioxidants, and/or cryoprotectants.

A further embodiment comprises a kit, comprising a pharmaceutical dosage unit comprising a therapeutically effective amount of a composition as described herein, and optionally further comprising a vehicle for dilution of the pharmaceutical composition. In another aspect, the kit may include instructions for use. In one illustrative kit, the Soli or other compounds described herein is present as a single dose, or multiple dose, or multiple dose concentrate. It is appreciated that the concentrate may be administered directly, or alternatively is further diluted into a diluent for administration.

It is to be understood that solithromycin, and other compounds described herein may be protonated in compositions and formulations having a pH less than 7. Accordingly, the methods, uses, compositions, and formulations described herein as comprising solithromycin, and/or other compounds described herein are understood to also comprise protonated forms of each of the foregoing.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carboxylic acid and derivatives thereof” includes the group CO₂H and salts thereof, and esters and amides thereof, and CN.

As used herein, the term “sulfonic acid or a derivative thereof” includes SO₃H and salts thereof, and esters and amides thereof.

The term “optionally substituted” as used herein includes the replacement of one or more hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

Monosaccharides, or simple sugars, consist of a single polyhydroxy aldehyde or ketone unit. Representative monosaccharides include, by way of illustration only, hexoses such as D-glucose, D-mannose, D-xylose, D-galactose, L-fucose, and the like; pentoses such as D-ribose or D-arabinose and ketoses such as D-ribulose or D-fructose. Disaccharides contain two monosaccharide units joined by a glycosidic linkage. Disaccharides include, for example, sucrose, lactose, maltose, cellobiose, and the like. Oligosaccharides typically contain from 2 to 10 monosaccharide units joined by glycosidic linkages.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

Pharmaceutically acceptable salts of the compounds described herein may be formed from one or more of the following illustrative acids, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (-L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (-L), salicylic acid, sebacic acid, stearic acid, succinic acid, sulfuric acid, tartaric acid (+L), thiocyanic acid, toluenesulfonic acid (p), undecylenic acid, and the like.

The terms “effective amount” and “therapeutically effective amount” as used herein, refer to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

The formulations described herein have been observed to achieve therapeutically effective concentrations in the target tissues or regions of the eyes, including the aqueous humor, cornea, conjunctiva, eyelids, and tears. Without being bound by theory, it is believed herein that therapeutically effective concentrations in the tears, conjunctivae, lids, cornea and aqueous humor indicates efficacy in treating dry eye diseases, including dry eye diseases accompanied by conjunctivitis, neonatal conjunctivitis, blinding trachoma, and the like, and/or accompanied by inflammation. It is to be understood that therapeutically effective concentrations are achieved by either a Cmax or AUC that is effective against the underlying target organism. The compounds and formulations described herein have demonstrated unexpectedly high AUC, especially due to significant concentrations in the target tissue(s) even at 12 h post administration. The compounds and formulations described herein have also demonstrated unexpectedly rapid corneal penetration. It is to be understood that low plasma, or other systemic levels, are desirable when the formulations described herein are administered topically to the eye. Without being bound by theory, it is believed herein that sustained exposure in the tears indicates sustained efficacy. In addition, concentrations at later timepoints after addition indicate that the active ingredients entered target tissues and cells in high concentrations before being washed away during naso-lacrimal drainage from the eye, and following entry is being slowly released. That slow release indicates long exposure times, and also a sustained bathing of the surface of the eye with active ingredients.

In another embodiment, a method for the treatment of one or more dry eye diseases, or a disorder related to one or more dry eye diseases, comprising the step of administering to a subject in need thereof a therapeutically effective amount of a pharmaceutical composition adapted for topical administration comprising Soli or a related compound is described herein.

In another embodiment, a use of a pharmaceutical composition adapted for topical administration comprising Soli or a related compound, as described herein, for the treatment of one or more dry eye diseases, or a disorder related to one or more dry eye diseases, is described herein.

In another embodiment, a pharmaceutical composition adapted for topical administration comprising Soli or a related compound, as described herein, for the manufacture of a medicament for the treatment of one or more dry eye diseases, or a disorder related to one or more dry eye diseases, is described herein.

In another embodiment, a method or use described above is one wherein the subject is a mammal, a fish, a bird or a reptile. As another embodiment, there is provided a method or use wherein the subject is a mammal. As another embodiment, there is provided a method or use wherein the subject is a human.

Topical administration means the application, directly to the surface of an eye, of a composition described herein. In an illustrative embodiment, the composition is applied directly to an eye as a single dose (equivalent to a dose in the range from about 1 mg to about 10 mg, from about 1 mg to about 5 mg, from about 1 mg to about 4 mg, from about 1 mg to about 3 mg, from about 1 mg to about 2 mg) per day for 5 to 14 days, or for 5 to 7 days, or for about 5 days. It is to be understood that such single doses referred to herein generally mean the amount for a single eye.

It is to be understood that references made herein to water as part of the methods, uses, compositions, and formulations described herein, generally refer to sterile water, suitable for use, such as water for injection, ultrapure water, and the like.

The following examples further illustrate specific embodiments of the invention; however, the following illustrative examples should not be interpreted in any way to limit invention. Alternative formulations of the compounds and compositions are described in PCT International Application No. PCT/US2018/018523, the disclosures of which are incorporated herein by reference.

### EXAMPLES

EXAMPLE. Immortalized human meibomian gland epithelial cells (HMGECs) are cultured in the presence of vehicle or test compound, such as Soli (2, 10, or 20 μg/ml) for 1 to 7 days under proliferating (keratinocyte serum-free medium; KSFM) or differentiating (DMEM/F12 plus 10% fetal bovine serum) conditions. A positive control (epidermal growth factor and bovine pituitary extract for proliferation; Azi for differentiation) and a negative control (media only) are included with the experiments. Additional details are described in JAMA Ophthalmol 132(2): 226-228 (Feb. 1, 2014) and Current Eye Research, 1-6 (2017), each of which is incorporated herein by reference.

HMGECs are evaluated for cell number, neutral lipid content (LipidTox) and lysosome accumulation (LysoTracker). Compounds described herein, such as Soli, induce a rapid and dose-dependent increase in the accumulation of neutral lipids and lysosomes in HMGECs. Strong lysosomal effects are observed with the 10 μg/ml dose of Soli (relative stimulation=30), on the first day of dosing, and those effects are statistically different (p<0.01) from the control group (relative stimulation=13). In contrast, the 10 μg/m1Azi dose is not statistically different from the control group (relative stimulation=18) until day 3. The effects of Soli and Azi on HMGEC differentiation were observed to be similar after 3 days of culture. Soli does not cause unwanted proliferation of HMGECs during a 7-day dosing period. Compounds described herein, such a Soli, affect lysosome and lipid accumulation and differentiation of HMGECs. The effect of Soli on lysosome appearance is faster than that of Azi. In related studies with other cell lines, Soli shows an IC50 for phospholipase inhibition (100 μM) that is comparable to gentamicin and amikacin, and is at least 3-fold better than other macrolides (Azi, 300 μM; Ery>400 μM). In addition, the phospholipase inhibition by Soli is rapidly reversible; whereas inhibition by Azi remains unchanged for several days after dosing is stopped.

EXAMPLE. Ocular pharmacokinetics in rabbits is measured by LC/MS/MS analysis of Soli concentrations in tears, cornea, aqueous humor, conjunctiva, and eyelids at 0.1-24 hours after topical administration of several Soli ophthalmic formulations. The formulations described herein exhibit high tissue exposure in target tissues, and very low systemic exposure (plasma). Soli penetrates the cornea and ocular surface tissues, resulting in effective intraocular concentrations as well as sustained levels in ocular surface tissues and tears for up to 12 hours after dosing.

| | Formulation 1 | Formulation 3 | Formulation 4 |
|---|---|---|---|
| | (single dose) | (single dose) | (single dose) |
| Tissue | Cₘₐₓ/AUC | Cₘₐₓ/AUC | Cₘₐₓ/AUC |
| Tears | 488,000/241,000 | 1,830,000/812,000 | 679,000/350,000 |
| Conjunctiva | 16,000/11,400 | 19,500/14,800 | 52,000/93,600 |
| Cornea | 18,600/54,800 | 39,300/95,200 | 28,000/165,000 |
| Eyelid | 22,100/56,200 | 42,100/134,000 | 40,700/209,000 |
| Aqueous | 46.5/452 | 77.7/606 | 71/611 |
| Humor | | | |
| Plasma | 7.85/9.2 | 5.69/5.32 | 7.35/NC |

NC = not calculated.
PK Parameter values are expressed in ng/mL for Cₘₐₓ and ng · h/mL for AUC.
Repeated dosing did not result in tissue accumulation.
Dosing 3 times per day for 3 days gave equivalent PK values.

EXAMPLE. The formulations described herein maintain therapeutically relevant concentrations in target tissues for up to 12 hours. It was unexpectedly observed that, exposure to target tissues was sustained for 12 hours after administration.

| | Formulation 3 |
|---|---|
| | (single dose) |
| Tissue | C_{12 h} |
| Tears | 39,600 |
| Conjunctiva | 53,800 |
| Cornea | 126,000 |
| Eyelid | 96,000 |
| Aqueous Humor | 4.6 |
| Plasma | 0 |

EXAMPLE. Corneal Permeability. Corneal permeability is determined using conventional protocols. Briefly, corneal permeability is determined on freshly excised bovine (calf) cornea using a Franz-Cell Diffusion Apparatus generally with n=3/group. Freshly excised calf cornea are stored until use in hydrating solution containing glutathione and buffer.

The Apparatus includes a donor chamber on the top where a predetermined volume of the formulation is pipetted and a jacketed receptor cell with a sampling side arm. The joint between the donor and receptor cell is upward-convex, mimicking the shape of the cornea. The corneal membrane is placed on the ball joint with the cornea facing the donor chamber. The Apparatus is clamped to secure the cells with the donor and receptor chambers aligned. Each cell is placed in one of the slots of the temperature-controlled cell holder. The cell holder consists of 6 in-line jacketed cells mounted on a single unit with individual magnetic stir plates, with each cell connected to the main system water jacket. The jacket is maintained at 37° C. for the duration of the experiment using a recirculating heating bath. Each receptor cell holds 5 mL of sink solution, and each donor cell holds 200 μL of the formulation being studied.

The receptor fluid, such as 1% HP-β-CD in a pH 7 phosphate buffer, is added to each cell using a syringe until there is a convex meniscus on the donor cell joint. The volume is recorded. After weighing the cornea, it is placed on top of the receptor-donor cell joint using a pair of forceps, ensuring that there are not any folds in the cornea or bubbles blocking the permeation port. Once in place, the donor cell caps are attached and locked in place with a metal clip.

Test samples are added in rapid succession by depositing 200 μL of the formulation into each donor chamber using a calibrated pipet and the times recorded. The donor chamber and sampling arm are sealed with parafilm or an equivalent material (caps) to ensure no significant evaporation occurs. Using a 100 μL pipette, 300 μL samples are withdrawn from each cell over 24 hours at 2, 4, 6, 7 and 22 hours, and transferred to HPLC vials for quantification by HPLC.

Flux (J) is the amount of test compound crossing the membrane per unit time. It is given in units of mass/area/time. Flux can be calculated by the formula: J=Q/(A·t), where Q is the quantity (micrograms) of compound traversing the membrane in time t (minutes), and A is the area of exposed membrane in cm². The units for flux are weight (micrograms)/cm²/minute. After completion of the diffusion, corneas are weighed to determine the quantity of bound test compound in the cornea, and the corneal thicknesses of each cornea is measured at the point of diffusion (the center) using Vernier calipers.

Formulated compounds are tested for their transverse diffusive ability through the membrane. Diffusion through biological membranes is directly correlated to the formulation excipients, its physical state (suspension, solution, emulsion, etc.) and its log P. For ease of passage through the corneal membrane, ideal log P is reportedly 2-3. For compounds with log P>3, the compound typically permeates the lipid epithelium of the cornea, only to be hindered by the hydrophilic stroma. For an indication like blepharitis, high corneal concentrations achieved with repeat dosing will result in a drug depot, acting like a sustained release system. Illustratively, CEM101 has a log P of 4.2 which results in high corneal and ocular tissue concentration, resulting in an increased uptake upon repeated dosing.

EXAMPLE. CEM101 Permeation. Both Formulation 1 and 2 showed comparable steady state diffusion rates, and total cumulative drug over time. Both Formulation 1 and 2 showed comparable corneal concentrations at t=22 h of 9.2% and 12%, respectively. Both Formulation 1 and 2 showed comparable flux of 0.33 μg/cm²/min and 0.40 μg/cm²/min, respectively. In addition, the formulations demonstrated a more rapid corneal penetration rate than Azasite. Soli was observed in receptor fluid at therapeutically effective concentrations (>1 μg/mL) within 1 h of administration. In contrast, Azasite was not observed until 4 h after administration.

EXAMPLE. Compounds described herein exhibit high cellular uptake and intracellular activity. Without being bound by theory, it is believed herein that the greater intracellular concentration and tissue concentration, and/or the faster speed of tissue uptake is at least partially responsible for the higher potency.

EXAMPLE. The compounds described herein exhibit intracellular localization and tissue distribution and concentration that is compatible with q.d. or once-a-day dosing. Soli was 50-fold and 100-fold more potent than azithromycin against phagocytized L. monocytogenes and L. pneumophila.

EXAMPLE. Compounds described herein exhibit consistent activity over a wide pH range. Compounds described herein exhibit consistent activity in the presence of serum. Compounds described herein exhibit low protein binding. Soli maintains its potency over a wider range than conventional compounds such as azithromycin, telithromycin, and clarithromycin. Soli undergoes only a 2-fold change in MIC in the presence of 10% serum. Soli exhibits low, 86%, protein binding in plasma. On the eye surface, it has been surprisingly discovered that the protein binding is not significant in that the MICs observed for Soli are also observed in vivo.

EXAMPLE. The following formulations are described:

| | Formulation | Formulation | Formulation | Formulation |
|---|---|---|---|---|
| | 1 | 2 | 3 | 4 |
| Components | % w/w | % w/w | % w/w | % w/w |
| Solithromycin | 1 | 1 | 1 | 1 |
| Boric Acid | 0.10 | 0.15 | 0.13 | 0.15 |
| EDTA | — | 0.05 | 0.04 | 0.05 |
| PEG35-Castor | 5 | 5 | 5 | 5 |
| Oil | | | | |
| PEG40- | 7 | 7 | 7 | 7 |
| Stearate | | | | |
| PEG400 | 1 | 1 | 1 | 1 |
| Citric Acid | 0.18 | 0.18 | 0.16 | 0.21 |
| Sodium citrate | 0.88 | 0.88 | 0.83 | 0.68 |
| BAK | — | — | 0.005 | 0.005 |
| Xanthan gum | — | — | 0.225 | 0.60 |
| HPLC water | QS | QS | QS | QS |
| Total | 100 | 100 | 100 | 100 |

Each of the formulations had an osmolality in the range from about 320 to about 335 mOsm.

EXAMPLE. The formulations described herein may have varying viscosities.

| | Formulation | Formulation | Formulation | Formulation |
|---|---|---|---|---|
| | 1 | 2 | 3 | 4 |
| Viscosity | 3 | — | 334 | 1539 |
| (cPs) | | | | |

EXAMPLE. Formulation Stability. To measure storage stability of the resulting formulation, approximately 5 mL aliquots of the completed formulation are transferred to Rexam 10 mL LDPE bottles, purged with nitrogen, and stored at 5° and 25° C. Stability of the Soli and formulation is measured at time zero, and at 3, 6, 9 and 12 month time points. The formulations described herein including boric acid have been unexpectedly found to exhibit high long-term and storage stability at multiple. Each of Formulations 1-4 were stable for >6 months at 5° C. and 25° C. At 5° C., total impurities remained much less than 2% by weight. At 25° C., total impurities remained much less than 3% by weight. At both temperatures, solithromycin assay remained well within 10% of its initial value.

EXAMPLE. The formulations described herein do not exhibit irritation in a conventional rabbit eye irritation test. Rabbits were dosed with each of Formulations 1-4 four times daily for 3 days according to a conventional ocular exposure assay. No signs of ocular redness, discomfort, or irritation were observed.

EXAMPLE. A 0.5 weight percent Soli ophthalmic solution is prepared by dissolving 50 g of Soli (0.5 weight %), 67.0 g (0.67 weight percent) boric acid, 20.7 g (0.207 weight percent) sodium borate decahydrate, 100 g (1.0 weight percent) glycerin, 100 g of polyethylene glycol 300 (1.0 weight percent), and 0.40 g (0.004 weight percent) thimerosal (as a preservative) in about 8000 g of deionized distilled water. The pH is adjusted to 7.2 with HCl and NaOH. The final batch weight is brought to 10,000 g with the addition of the required amount of water. The final solution is filtered through a 0.2 micron Millipore filter and filtered into vials.

EXAMPLE. The following composition is prepared (by w/w): Soli 3.50, Chlorbutol BP 0.50, Citric acid monohydrate 0.117, Sodium citrate dihydrate 0.112, Sodium citrate 1% solution qs, Hydroxypropylmethylcellulose 3.80, 2906 USP 4000 cps (sterile) Water to 100.00. Citric acid, sodium citrate and chlorbutol BP are dissolved in 95% of the total water and the solution sterilized. Soli is dispersed in the solution at ambient temperature using a high shear mixer. The hydroxypropylmethylcellulose, previously sterilized, is dispersed in the suspension and then allowed to hydrate over a period of about 15 minutes. The pH is adjusted to between 46 with a 1% solution of sterilized sodium citrate. The gel is adjusted to final weight with water and mixed thoroughly.

EXAMPLE. Compounds described herein exhibit potent anti-inflammatory activity. Cells. The human monocytic cell line U937 was obtained from the American Type Culture Collection (ATCC, Rockville, Md.). PBMCs from COPD patients were obtained from Brompton hospital and separated by AccuSPIN (Sigma-Aldrich). Cells were cultured in complete growth medium (RPMI 1640) (Sigma-Aldrich) supplemented with 10% fetal bovine serum (FBS) and 1% L-glutamine at 37° C. in a humidified atmosphere with 5% CO₂. U937 cells were differentiated into adherent macrophage-like morphology by exposure to PMA (50 ng/ml) for 48 h in complete growth medium. Cell viability was assessed microscopically by trypan blue staining. Cell toxicity was determined by MTT assay as needed. This study was approved by the ethics committee of the Royal Brompton Hospitals, and all subjects gave written informed consent.

Cell Lysis. Whole cell extracts were prepared as previously described (Kobayashi et al., 2011). Briefly, cell protein extracts were prepared using modified RIPA buffer (50 mM Tris HCL pH 7.4, 0.5% NP-40, 0.25% Na-deoxycholate, 150 mM NaCl with freshly added complete protease inhibitor cocktail (Roche, Mannheim, Germany)). Protein concentration was determined using the BCA Protein Assay (Thermo Fisher Scientific, Waltham, Mass.).

Cytokine ELISA. TNFα and IL-8 concentrations in the supernatant of cell cultures were determined by sandwich ELISA according to the manufacturer's instructions (R&D Systems Europe, Abingdon, UK).

Zymography. MMP9 enzyme activity was measured by gelatin zymography. Cell culture supernatants were diluted with equal amount of Laemli sample buffer (Bio-Rad, Hertfordshire, UK) and loaded on a Novex® 10% Zymogram (Gelatin) gel (Invitrogen Ltd, Paisley, UK). After electrophoresis, gels were incubated and rinsed with Novex® zymogram renaturing buffer (Invitrogen) for 30 min at room temperature. The gels were then rinsed in Novex® zymogram developing buffer (Invitrogen) for 30 min at room temperature prior to overnight incubation in the developing buffer at 37° C. After incubation, the gels were stained using a Colloidal Blue Staining Kit (Invitrogen) to visualize the zymogen bands.

NF-κB activity. The activation of NF-κB (p65 binding activity to NF-κB binding sequence) was determined using a TransAM NF-κB p65 Assay kit (Active Motif, Inc., Carlsbad, Calif.) according to the manufacturer's instruction. As shown above, whole cell extracts were prepared from PMA-differentiated U937 cells, and 20 μl of each extract was used for this study. Results were determined by measuring the spectrophotometric absorbance at 450 nm with a reference wavelength of 655 nm.

Statistical analysis. The results were expressed as the mean±SEM. Comparisons of data in two groups were performed using the Student's t test or the Wilcoxon signed rank test. Multiple comparisons were made by one-way ANOVA with post hoc test (Dunnett's) as appropriate. The difference was considered significant at p<0.05. IC50 values (50% inhibitory concentration) for macrolides for production of cytokines or MMP9 were calculated using Prism 4.0 (GraphPad Software Inc., San Diego, Calif.).

Anti-inflammatory effects of solithromycin in U937 cells. LPS significantly increased TNFα and IL-8 production in PMA-differentiated U937 cells (TNFα, 63.1±2.6 fold in LPS vs. non-stimulated; and CXCL8, 2.0±0.1 fold in LPS vs. non-stimulated cells, n=3). Solithromycin significantly inhibited both TNFα and CXCL8 at 100 μM. Although clarithromycin showed modest effects on both TNFα and IL-8 production at a higher concentration (333 μM), erythromycin and azithromycin did not inhibit them. Telithromycin at 100 μM did not inhibit production of TNFα and CXCL8. The IC₅₀ values for solithromycin on TNFα and CXCL8 release were 41.6±1.9 μM and 78.2±9.5 μM, respectively, and were superior to those for clarithromycin (IC50, 426.3±63.9 μM for TNFα and 506.5±44.0 μM for CXCL8).

The effects of macrolides on MMP9 activity, which was clearly elevated by PMA stimulation in U937 cells (9.9±2.0 fold in PMA vs. non-stimulation, n=3) is measured. Solithromycin remarkably reduced MMP9 activity, with an IC₅₀ of 14.9±3.1 μM. In contrast, clarithromycin and azithromycin showed 10-fold lower inhibitory effects than solithromycin whereas erythromycin showed no effect. Telithromycin also inhibited MMP9 activity, although to lesser extent than solithromycin, with an IC50 of 97.9 μM.

EXAMPLE. Formulations described herein are more efficacious than conventional compounds, such as azithromycin (Azi) against many pathogenic bacteria. Soli is 8-16 fold more potent than Azi. Soli exhibits a broader spectrum of antibacterial activity than Azi. Soli is active against all Azi resistant strains tested. Soli exhibits 10 fold greater tissue distribution than Azi both in terms of speed of uptake and ultimate tissue concentration. Soli exhibits 10 fold greater activity than conventional macrolides, and 50-100 fold greater activity against phagocytized L. monocytogenes and L. pneumophila. Soli exhibits 100-200 fold greater activity at acidic pH than Azi, including in L. monocytogenes and S. aureus. Soli exhibits 10 fold greater intracellular activity than Azi. Soli exhibits a wide therapeutic window for safety. Soli exhibits greater antibacterial potency in inflamed tissues. Soli exhibits greater anti-inflammatory properties than Azi. Soli exhibits greater solution stability than Azi.

| Organism | Solithromycin | Azithromycin |
|---|---|---|
| (# strains) | MIC90 (μg/ml) | MIC90 (μg/ml) |
| Streptococcus Pneumoniae | 0.25 | >16 |
| (150) | | |
| Streptococcus Pyogenes | 0.03 | >16 |
| (100) | | |
| Haemophilus influenzae | 2 | 2 |
| (100) | | |
| Chlamydophila pneumoniae | 0.25 | 0.125 |
| (10) | | |
| Legionella pneumophila | ≤0.015 | 2 |
| (30) | | |
| Mycoplasma pneumoniae | 0.000125 | 0.0005 |
| (38) | | |

EXAMPLE. The formulations described herein show overall higher potency against ocular pathogens than commercial standards.

MICs (μg/ml) of Solithromycin and Comparator Drugs against P. acnes (51 species)

| Drug | MIC Range | MIC 50% | MIC 90% |
|---|---|---|---|
| Solithromycin (CEM-101) | ≤0.002-0.25 | 0.015 | 0.06 |
| Penicillin | ≤0.03-1 | ≤0.03 | 0.06 |
| Cefdinir | ≤0.015-0.12 | 0.03 | 0.12 |
| Cefixime | ≤0.03-0.5 | 0.25 | 0.5 |
| Cefpodoxime | ≤0.015-2 | 0.5 | 2 |
| Vancomycin | 0.12-1 | 1 | 1 |
| Azithromycin | ≤0.015->32 | 0.06 | 4 |
| Clarithromycin | ≤0.015-0.5 | ≤0.015 | 0.25 |
| Daptomycin | 0.5-8 | 2 | 4 |
| Doxycycline | ≤0.008-1 | 0.06 | 0.12 |
| Levofloxacin | 0.06-2 | 1 | 1 |
| Linezolid | ≤0.03-0.5 | 0.25 | 0.5 |
| Trimethoprim/Sulfa | 0.06/1.19-2/38 | 0.25/4.75 | 0.5/9.5 |

MICs (μg/ml) of Solithromycin and Comparator Drugs against Bacterial Species

| | MIC Range | MIC50 | MIC90 | S | I | NS |
|---|---|---|---|---|---|---|
| S. pneumoniae | | | | | | |
| (30 species) | | | | | | |
| Solithromycin | ≤0.001-0.25 | 0.002 | 0.12 | 100% | 0 | 0 |
| Azithromycin | 0.03->32 | 0.06 | 8 | 73% | 3% | 23% |
| Moxifloxacin | 0.015-0.25 | 0.12 | 0.25 | 100% | 0 | 0 |
| Tobramycin | 4-16 | 16 | 16 | — | — | — |
| H. influenzae | | | | | | |
| (30 species) | | | | | | |
| Solithromycin | 1-4 | 2 | 2 | 100% | 0 | 0 |
| Azithromycin | 1-4 | 2 | 2 | 100% | 0 | 0 |
| Moxifloxacin | 0.015-0.06 | 0.03 | 0.06 | 100% | 0 | 0 |
| Tobramycin | 2-8 | 4 | 4 | — | — | — |
| MSSA | | | | | | |
| (31 species) | | | | | | |
| Solithromycin | 0.004-64 | 0.015 | 0.03 | 94% | 0 | 6% |
| Azithromycin | 1->32 | 2 | >32 | 61% | 0 | 39% |
| Moxifloxacin | 0.015-2 | 0.06 | 0.12 | 94% | 0 | 6% |
| Tobramycin | 0.12-4 | 0.5 | 0.5 | 100% | 0 | 0 |
| MSRA | | | | | | |
| (30 species) | | | | | | |
| Solithromycin | 0.008->64 | 0.03 | 64 | 70% | 0 | 30% |
| Azithromycin | 2->32 | >32 | >32 | 10% | 0 | 90% |
| Moxifloxacin | 0.06->8 | 2 | 8 | 10% | 3% | 87% |
| Tobramycin | 0.25->64 | 1 | >64 | 67% | 3% | 30% |
| S. epidermidis | | | | | | |
| (30 species) | | | | | | |
| Solithromycin | 0.002->64 | 0.015 | 32 | 80% | 3% | 17% |
| Azithromycin | 0.25->32 | >32 | >32 | 37% | 0% | 63% |
| Moxifloxacin | 0.03->8 | 0.06 | 4 | 83% | 3% | 13% |
| Tobramycin | 0.12-64 | 0.5 | 32 | 73% | 3% | 23% |

S = susceptible, I = intermediate susceptibility, NS = not susceptible.

| | | | MIC (μg/mL) | | |
|---|---|---|---|---|---|
| Organism | N | Compound | MIC₅₀ | MIC₉₀ | Range |
| Corynebacterium spp. | 10 | CEM-101 | 0.015 | 0.5 | ≤0.008-16 |
| | | azithromycin | >16 | >16 | 0.12->16 |
| Haemophilus influenzae | 100 | CEM-101 | 1 | 2 | 0.12-4 |
| | | azithromycin | 2 | 2 | 0.25-4 |
| Streptococcus pneumoniae | 150 | CEM-101 | 0.015 | 0.25 | ≤0.008-0.5 |
| | | azithromycin | >16 | >16 | 0.03->16 |
| Staphylococcus aureus | 201 | CEM-101 | 0.12 | >16 | 0.03->16 |
| | | azithromycin | >16 | >16 | 0.5->16 |
| CA-MRSA | 30 | CEM-101 | 0.12 | 0.12 | 0.06-0.12 |
| | | azithromycin | >16 | >16 | >16 |
| Chlamydophila pneumoniae | 10 | CEM-101 | 0.25 | 0.25 | 0.25-1 |
| | | azithromycin | 0.125 | 0.125 | 0.015-0.125 |
| Chlamydia trachomatis | 10 | CEM-101 | 0.25 | 0.25 | 0.125-0.5 |
| | | azithromycin | 0.125 | 0.125 | 0.015-0.125 |
| Haemophilus parainfluenzae | 11 | CEM-101 | 2 | 2 | 1-2 |
| | | azithromycin | 1 | 2 | 0.5-2 |
| Legionella pneumophila | 30 | CEM-101 | ≤0.015 | ≤0.015 | ≤0.015 |
| | | azithromycin | 1 | 2 | 0.25-4 |
| Listeria monocytogenes | 10 | CEM-101 | 0.03 | 0.03 | 0.03 |
| | | azithromycin | 0.5 | 1 | 0.5-1 |
| Moraxella catarrhalis | 21 | CEM-101 | 0.12 | 0.12 | ≤0.008-0.5 |
| | | Azithromycin | 0.03 | 0.06 | 0.03-0.5 |
| Mycobacterium avium | 30 | CEM-101 | 1 | 1 | 1 |
| | | azithromycin | 16 | 16 | 8->512 |
| Mycoplasma hominis | 13 | CEM-101 | 0.004 | 0.008 | 0.002-0.008 |
| | | azithromycin | 4 | 2 | 0.5-4 |
| Mycoplasma pneumoniae | 38 | CEM-101 | 0.000032 | 0.000125 | ≤0.000000063-0.5 |
| | | azithromycin | 0.00025 | 0.0005 | ≤0.000016-≥32 |
| Neisseria gonorrhoeae | 34 | CEM-101 | 0.06 | 0.12 | 0.03-0.25 |
| | | azithromycin | 0.25 | 0.5 | 0.06-2 |
| Peptostreptococcus spp. | 10 | CEM-101 | 0.06 | 0.25 | ≤0.03-0.25 |
| | | azithromycin | 8 | >64 | 2->64 |
| Ureaplasma urealyticum | 10 | CEM-101 | 0.008 | 0.031 | 0.004-0.063 |
| | | azithromycin | 2 | 4 | 0.5-4 |
| Viridans group streptococci | 51 | CEM-101 | ≤0.008 | 0.06 | ≤0.008-0.12 |
| | | azithromycin | 0.12 | 4 | ≤0.008-16 |
| Streptococcus mitis | 73 | CEM-101 | ≤0.03 | 0.06 | ≤0.03-0.25 |
| Streptococcus pyogenes | 407 | CEM-101 | ≤0.03 | ≤0.03 | ≤0.03-0.5 |
| | 100 | azithromycin | >16 | >16 | — |
| Streptococcus pyogenes (a) | 407 | CEM-101 | ≤0.03 | ≤0.03 | ≤0.03-0.5 |
| Streptococcus agalactiae | 535 | CEM-101 | ≤0.03 | ≤0.03 | ≤0.03-0.5 |
| Streptococci (Groups C, F, G) | 185 | CEM-101 | ≤0.03 | ≤0.03 | ≤0.03-0.25 |

1. A method for treating one or more dry eye diseases in a host animal, the method comprising the step of topically administering to an eye of the host animal an effective amount of a composition comprising one or more compounds of the formula [chemical structure]
or a salt thereof, wherein:
R¹⁰ is hydrogen or acyl;
X and Y are taken together with the attached carbon to form carbonyl;
V is C(O);
W is H, F, Cl, Br, I, or OH;
A is CH₂, C(O), C(O)O, C(O)NH, S(O)₂, S(O)₂NH, or C(O)NHS(O)₂;
B is Co to Cio alkylene, C2 to C10 alkenylene, C2 to C10 alkynylene or C4 to C10 alkenylalkynylene; and
C is aryl, heteroaryl, arylalkyl, or heteroarylalkyl, each of which is optionally substituted.

2. (canceled)

3. (canceled)

4. The method of claim 1 wherein the disease is keratoconjunctivitis sicca.

5. The method of claim 1 wherein the disease is dry eye syndrome.

6. The method of claim 1 wherein the disease is keratitis sicca.

7. The method of claim 1 wherein the disease is keratoconjunctivitis.

8. The method of claim 1 wherein the disease is blepharitis.

9. The method of claim 1 wherein the disease is blepharoconjunctivitis.

10. The method of claim 1 wherein the compound is solithromycin, or a salt thereof.

11. The method of claim 1 comprising boric acid or a salt thereof.

12. The method of claim 11 further comprising a metal chelating agent.

13. The method of claim 1 comprising one or more polyethylene glycol esters.

14. The method of claim 13 wherein the one or more polyethylene glycol esters are selected the group consisting of PEG castor oils and PEG stearates, and combinations thereof.

15. The method of claim 1 comprising one or more polyethylene glycols.

16. The method of claim 1 comprising xanthan gum.
